(19) United States
(12) Patent Application Publication
(10) Pub. No.: US 2004/0137466 A1
(43) Pub. Date: Jul. 15, 2004

(54) Methods of isolating and/or identifying related plant sequences

(76) Inventors: Diane K. Jofuku, Oak Park, CA (US); Anne-Marie Bouckaert, Alsemberg (BE)

Correspondence Address: BIRCH STEWART KOLASCH & BIRCH, PO BOX 747, FALLS CHURCH, VA, 22040-0747, US

(21) Appl. No.: 10/642,277

(22) Filed: Aug. 18, 2003

Related U.S. Application Data
(63) Continuation of application No. 09/512,882, filed on Feb. 25, 2000, now abandoned.
(60) Provisional application No. 60/121,700, filed on Feb. 25, 1999.

(51) Int. Cl. C12Q 1/68

(52) U.S. Cl. 435/6

(57) ABSTRACT

The invention provides an improved method for isolating or characterizing genes in a second or target plant species that have substantial sequence identity to at least a portion of a gene in a first or template plant species.

This application is a continuation of co-pending application Ser. No. 09/512,882 (Attorney No. 2750-0198P), filed on Feb. 25, 2000, the entire contents of which are hereby incorporated by reference. Through application Ser. No. 09/512,882, this application also claims priority under 35 USC §119(e) of the following provisional application, the entire contents of which are hereby incorporated by reference:

| Country | Filing Date | Attorney No. | Application No. |
|---|---|---|---|
| United States | Oct. 25, 1999 | 2750-0117P | 60/121,700 |

### FIELD OF THE INVENTION

This invention is related to utilizing molecular biology and recombinant DNA technology to isolate and/or identify sequences from different plant families.

### BACKGROUND OF THE INVENTION

References describing codon usage include: Carels et al., J. Mol. Evol., Vol. 46, pp. 45-53 (1998) and Fennoy et al., Nucl. Acids Res., Vol. 21, No. 23, pp. 5294-5300 (1993).

AP2 like proteins and genes of Arabidopsis re described in copending U.S. application Ser. Nos. 08/700,152; 08/879,827; 08/912,272; and 09/026,039.

### SUMMARY OF THE INVENTION

The present invention relates to a method of isolating a target polynucleotide from a target plant species that encodes a polypeptide exhibiting a desired degree of sequence identity to a conserved region of a template polypeptide from a template plant species. The method comprises:

(a) identifying the amino acid sequence of the conserved region in the template polypeptide;

(b) generating an oligonucleotide comprising a sequence wherein the sequence or its reverse complement encodes at least four amino acids of the conserved region identified in step (a), wherein

(i) the nucleotide of the first and second position of at least three codons are the same as the corresponding nucleotides in the template polynucleotide encoding the template polypeptide; and

(ii) the nucleotide of the third position of the codon of step (i) is the same as the nucleotide at the third position of the most preferred codon of the second plant class, family, genera, or species for that amino acid in the portion of the conserved region;

further wherein the oligonucleotide preferably does not comprise homopolymers of more than four nucleotides; and the oligonucleotide is not degenerate;

(c) providing a composition comprising the target polynucleotide;

(d) contacting the oligonucleotide and the target polynucleotide under conditions that permit hybridization and formation of a duplex.

Identification of target polynucleotide can be accomplished by detection of the duplex of step (d) . Further, both single stranded and double stranded target polynucleotides can be generated from the duplex of step (d).

### DETAILED DESCRIPTION OF THE INVENTION

Definitions

The usage of the term “plant family” herein refers to the common nomenclature used to classify organisms, for example Liliaceae and Orchidaceae are plant families.

General Method

The present invention relates to a method of isolating and/or identifying genes in nucleic acids from a target plant species related to a gene or corresponding cDNA or other nucleic acids from a template plant species. Preferably, the target and template plant species are from different plant families.

In another embodiment of the invention, the method includes identifying and/or isolating from a target plant species a target polynucleotide that encodes a conserved region that exhibits at least 70% sequence a conserved region encoded by the template polynucleotide from another plant species.

The target and template polynucleotides can be either RNA or DNA or derivatives thereof. The oligonucleotides to be utilized can be RNA, DNA, or derivatives thereof, such as protein-nucleic acids, (PNAs). The target polynucleotide can be isolated from cDNA or genomic libraries or fixed on microarrays and need not be isolated directly from the second or target plant organism. Such plant sequences can be first subcloned into intermediary vectors or organisms.

The method utilizes sequences from a conserved region of the polypeptide encoded by the template polynucleotide. A “conserved region” is a primary sequence within a polypeptide that correlates to an in vitro activity, in vivo activity, or a secondary structure. For example, the active site of a serine protease exhibits a particular tertiary structure that is responsible for the activity of the protein. That same tertiary structure can be encoded by way of different amino acid sequences, but certain portions of the sequence tend to be the same among the variants. The amino acid sequence identity of conserved regions from related proteins can be as low as approximately 35%. Thus, even polypeptides that exhibit about 35% sequence identity can be useful to identify a conserved region. More typically, such conserved regions of related proteins exhibit at least 50% sequence identity; even more typically at least about 60%; even more typically, at least 70% sequence identity, more typically at least 80%, even more typically about 90% sequence identity.

A. Identifying Conserved Regions

Conserved regions can be identified by locating a primary sequence within the template polypeptide that:

(i) is a repeated sequence;

(ii) forms some secondary structure, such as helices, beta sheets, etc.

(iii) establishes positively or negatively charged domains;

(iv) represent a protein motif or domain. See, for example, the Pfam web site describing the consensus sequence for a variety of protein motifs and domains. The sites on the World Wide Web in the UK at http://www.sanger.ac.uk/Pfam/ and in the US at http://genome.wustl.edu/Pfam/. For a description of the information included at the Pfam database, see Sonnhammer et al., Nucl Acids Res 26(1): 320-322 (Jan. 1, 1998); and Sonnhammer E L, Eddy S R, Durbin R (1997) Pfam: A Comprehensive Database of Protein Families Based on Seed Alignments, Proteins 28:405-420; Bateman et al., Nucl. Acids Res. 27(1):260-262 (Jan. 1, 1999); and Sonnhammer et al., Proteins 28(3):405-20 (July 1997).

From this database, consensus sequences of protein motifs and domains can be aligned with the template polypeptide sequence to determine the conserved region.

In addition, conserved regions can be determined by aligning sequences of the same or related genes in closely related plant species. Closely related plants species preferably are from the same family. Alternativelly, plant species that are both monocots or both dicots are preferred.

Sequences from two different plant species are adequate. For example, sequences from Canola and Arabidopsis can be used to identify the conserved region. Such related polypeptides from different plant species need not exhibit an extremely high sequence identity to aid in determining conserved regions.

Even polypeptides that exhibit about 35% sequence identity can be useful to identify a conserved region. More typically, such conserved regions of related proteins exhibit at least 50% sequence identity; even more typically at least about 60%; even more typically, at least 70% sequence identity, more typically at least 80%, even more typically about 90% sequence identity.

Typically, the conserved region of the target and template polypeptides or polynucleotides exhibit at least 70% sequence identity; more preferably, at least 80% sequence identity; even more preferably, at least 90% sequence identity; most preferably at least 92, 94, 96, 98, or 99% sequence identity. The sequence identity can be either at the amino acid or nucleotide level.

Sequence identity can be determined by optimal alignment of sequences to compare by the local homology algorithm of Smith and Waterman, Add. APL. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used. “Percentage of sequence identity” is determined by comparing two optimally aligned sequences over a comparison window, wherein the portion of the polynucleotide sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Alternatively, the polynucleotides of a conserved region of closely related species will hybridize under stringent conditions wherein one of the polynucleotides is a probe to determine the conserved region. “Stringency” is a function of probe length, probe composition (G+C content), and hybridization or wash conditions of salt concentration, organic solvent concentration, and temperature. Stringency is typically compared by the parameter “Tₘ”, which is the temperature at which 50% of the complementary The relationship of hybridization conditions to Tₘ (in ° C.) is expressed in the mathematical equation

Tₘ=81.5−16.6(log₁₀[Na⁺])+0.41(%G+C)−(600/N) (1)

where N is the length of the probe. This equation works well for probes 14 to 70 nucleotides in length that are identical to the target sequence. For probes of 50 nucleotides to greater than 500 nucleotides, and conditions that include an organic solvent (formamide) an alternative formulation for Tₘ of DNA-DNA hybrids is useful.

Tₘ=81.5+16.6 log {[Na^{+]}/(1+0.7(Na⁺])}+0.41(%G+C)−500/L 0.63(%formamide) (2)

where L is the length of the probe in the hybrid. (P. Tijessen, “Hybridization with Nucleic Acid Probes” in Laboratory Techniques in Biochemistry and Molecular Biology, P. C. vand der Vliet, ed., c. 1993 by Elsevier, Amsterdam.) With respect to equation (2), Tₘ is affected by the nature of the hybrid; for DNA-RNA hybrids Tₘ is 10-15° C. higher than calculated, for RNA-RNA hybrids Tₘ is 20-25° C. higher. Most importantly for use of hybridization to identify DNA including genes corresponding to a template sequence, Tₘ decreases about 1° C. for each 1% decrease in homology when a long probe is used (Bonner et al., J. Mol. Biol. 81:123 (1973)).

Equation (2) is derived under assumptions of equilibrium and therefore, hybridizations according to the present invention are most preferably performed under conditions of probe excess and for sufficient time to achieve equilibrium. The time required to reach equilibrium can be shortened by inclusion of a “hybridization accelerator” such as dextran sulfate or another high volume polymer in the hybridization buffer.

When the practitioner wishes to examine the result of membrane hybridizations under a variety of stringencies, an efficient way to do so is to perform the hybridization under a low stringency condition, then to wash the hybridization membrane under increasingly stringent conditions. With respect to wash steps preferred stringencies lie within the ranges stated above; high stringency is 5-8° C. below Tₘ.

B. Generating an Oligonucleotide

Once a conserved region is identified, an oligonucleotide can be generated to isolate and/or identify a target sequence. This oligonucleotide is usually not degenerate. Preferably, the oligonucleotide comprises a sequence wherein it or its reverse complement encodes a portion of the conserved region.

The portion is at least 3 amino acids in length, more typically, 4 amino acids in length; more typically at least 6 amino acids, even more typically at least 10 amino acids. Usually, the portion is at least than 40 amino acids; more usually, at least 30 amino acids; even more usually, usually at least 20 amino acids in length. A preferred range is from 3 to 18 amino acids in length.

The choice of which portion of the conserved region to use is based on convenience. Preferably, the portion of the conserved region is chosen to minimize the number of amino acids that are encoded by four or more codons. For example, the number of alanines, arginines, glycines, leucines, prolines, serines, threonines, and valines is minimized.

The sequence of the oligonucleotide is designed using the following criteria:

(1) Amino acid sequence of the conserved region of a template polypeptide;

(2) Preferred codon usage in the class, family, genera, or species of target plant species; and

(3) Polynucleotide sequence of the template polypeptide.

Typically, the oligonucleotide comprises at least one codon wherein the first and second position of the codon is the same as the corresponding position in the template polynucleotide and the third position is the same as the third position of the most preferred codon.

This preferred codon can be the most preferred of the plant class from which the target plant species belongs. For example, if the target plant species belongs to the dicot class, the preferred codon can be the one that is preferred by all dicots. Alternatively, the preferred codon can be one preferred in the family, genera, or species that the target plant species belongs. (The terms class, family, genera, and species is used in accordance with the accepted classification system of all organisms.)

One example is illustrated below:

| | |
|---|---|
| Conserved Region (AA): | . . . Aaa₁ - Aaa₂ - Aaa_{3 . . .} |
| Template Polynucleotide | (N₁N₂N₃) - (N₄N₅N₆) - (N₇N₈N₉) |
| encoding conserved region: | |
| Preferred Codons for | (X₁X₂X₃) (X₄X₅X₆) (X₇X₈X₉) |
| conserved regions in | |
| target plant species: | |
| Oligonucleotide: | (N₁N₂X₃) - (N₄N₅X₆) - (N₇N₈X₉) . . . |

The third position of the second most preferred codon is utilized if the first two positions of the template polynucleotide do not match the most preferred codon, but the template polynucleotide matches the first two positions of the second most preferred codon.

Further, the oligonucleotide sequence is chosen to avoid homopolymers of more than four nucleotides. Preferably, a portion of the conserved region is chosen to prevent such homopolymers from occurring in the oligonucleotide. Homopolymers can be included in the oligonucleotide if such a stretch is found in the template sequence and is preferred by the target plant species codon usage.

A higher percentage of guanosines and cytosines are preferred in the oligonucleotide sequence when a monocot target polynucleotide is to be isolated or identified using a template polynucleotide from a dicot plant species. Thus, for example, a guanosine or cytosine is preferred at the third position of the codons in the oligonucleotide when isolating and/or identifying a target sequence from a monocot using an Arabidopsis sequence as a template polynucleotide.

In contrast, higher percentage of adenines and thymidines are preferred in the oligonucleotide sequence when a dicot target polynucleotide is to be isolated or identified using a template polynucleotide from a monocot plant species. Thus, for example, an adenosine or thymidine may be preferred at the third position of the codons in the oligonucleotide when isolating and/or identifying a target sequence from a dicot, such as Arabidopsis, using a monocot sequences from corn as a template polynucleotide.

Oligonucleotides of the invention are at least 12, 16, 18, 20, 25 30, 35, 40, 45 or even at least 50 nucleotides in length.

The sequence and length are chosen to generate an oligonucleotide that is capable of forming a detectable duplex with target nucleotides. The oligonucleotide can include additional nucleotides, for example inosine, that bind to sequences in the template that flank the portion of the polynucleotide encoding the conserved region to stabilize the formed duplex. Additional non-plant polynucleotide sequences may be helpful as a label to detect the formed duplex as a primary site for PCR or to insert a restriction site for later cloning of the isolated plant sequences.

More than one oligonucleotide can be generated from the conserved region to be used in the identification and isolation procedures.

C. Isolating and/or Identifying Target Polynucleotide Sequences

The target polynucleotide sequence is isolated by contacting the oligonucleotide of the invention with a composition that comprises the target polynucleotide under conditions that permit hybridization and formation of a duplex. The duplex is then detected and the target polynucleotide can be isolated.

Exemplary procedures for identifying and/or isolating target polynucleotides that can be used include polymerase chain reaction (PCR), Southern hybridization, and polynucleotide capture.

Isolation and/or identification of a target polynucleotide can be performed using any number of oligonucleotides constructed using the instant invention.

For example, a single probe can be used in colony hybridization assays to identify from of library of clones the particular clone or clones that contain the desired target sequence. Such techniques are known, for example, for bacterial, yeast, and viral clones. Further, a single probe can also be used to generate the target polynucleotides from a starting material comprising a plurality of polynucleotides, for example in a nick translation or cDNA synthesis or random priming or end labeling.

Single probes can be used in gel isolation techniques, such as Southern or Northern hybridization for identifying polynucleotides that correspond to the target polynucleotide to be isolated. For example, inserts of a cDNA library comprising the target polynucleotide are separated by length and are bound to a solid support so as to preserve the separation. Next, the oligonucleotide can be labeled and used to identify the fragments that hybridize to the oligonucleotide. Hybridization and wash stringency can be varied as defined above, but preferably stringent conditions are used.

Alternatively, a single oligonucleotide can be bound to a solid support to isolate the desired target polynucleotide. The solid support can be exposed to a plurality of polynucleotides. The solid support can capture those polynucleotides that hybridize to the oligonucleotide, and the unwanted polynucleotides can be washed away. The target polynucleotide can be released from the solid support and further characterized or inserted into a vector.

Other methods for capturing target polynucleotides to a solid support using an oligonucleotide are described in Li et al., U.S. Pat. No. 5,500,356; and Laffler et al., U.S. Pat. No. 5,858,652.

Oligonucleotides of the invention can be used as primers in PCR to amplify the desired target polynucleotide sequences from a plurality of polynucleotides, such as a sample of mRNA from a tissue or a cDNA library. The reaction is run using the oligonucleotides as primers and mRNA (or cDNA) or genomic DNA from the target species as a substrate. The PCR product can be inserted directly into a vector for further processing. Alternatively, gel electrophoresis or other separations can be performed on the PCR product and the target polynucleotide can be identified by Southern hybridization techniques for further characterization or final isolation.

Amplification methods using a single oligonucleotide based on the instant invention specific for the target polynucleotide can be used for isolation and/or identification. Such a technique is single-primer PCR (SPPCR). A description of the method is described in Screaton et al., Nucl. Acids Res. 21: 2263-2264 (1993).

Other methods of isolating target polynucleotides with a single gene specific primer are described in Frohman et al., Proc Natl Acad Sci USA 85(23):8998-9002 (December 1988) and Uematsu et al., Immunogenetics 34(3):174-8 (1991).

Also, non-specific primers comprising, for example, poly-A, poly-T, or cap sequences, can be used in conjunction with a specific oligonucleotide of the invention.

PCR amplification methods can be performed using either one or two specific oligonucleotides generated from the conserved region of the template polypeptide. Preferably, the primers generate a product that is longer than the total length of the primers. Typically, using two primers, the portions of the conserved regions that are encoded by the oligonucleotides or their reverse complements are separated by at least about 5 amino acids, more typically by at least about 30 amino acids, more typically by at least about 50 or 100 amino acids. In another acceptable arrangement, the oligonucleotides (or their reverse complements) each represent a portion of two different conserved regions of a single polypeptide. Then the polynucleotide between the conserved regions, perhaps inclusive of one, or both of them, is amplified.

Nested primers can be used to PCR amplify the target polynucleotide sequences.

Compositions and methods for reverse transcriptase-polymerase chain reaction (RT-PCR) is another means of isolating and/or identifying target polynucleotides utilizing oligonucleotide primers of the invention. See, for example Lee et al, WO9844161A1 by Applicant Life Technologies.

Other amplification techniques, such as rapid amplification of cDNA ends can be used to isolate full length genes. One such procedure is described in Fehr et al., Brain Res Brain Res Protoc 3(3):242-51 (January 1999).

D. Identifying Target Polynucleotides

The oligonucleotides of the invention can be utilized to identify the sequence of the target polynucleotides. For example, the oligonucleotides can be used in a modified PCR procedure to obtain the sequence of the target polynucleotide. See, for example, Mitchell et al., U.S. Pat. No. 5,817,797; Uhlen, U.S. Pat. No. 5,405,746; Ruano, U.S. Pat. No. 5,427,911; Leushner et al, U.S. Pat. No. 5,789,168; and

The isolated target polynucleotide can be used in any sequencing procedure, such as the known dideoxy termination method and its modifications, to identify the specific sequences.

E. Further Isolation of Target Polynucleotides

When the sequence of the target polynucleotides is identified, primers can be constructed using sequence from the very termini of the target polynucleotides to “primer walk” and obtain the remaining sequences of the gene of which the target polynucleotides are a portion. See, for example, Screaton et al., Nucl. Acids Res. 21: 2263-2264 (1993).

The target polynucleotide can also be used to identify clones or colonies in a library that comprise sequences from the same gene as the target polynucleotide.

Plant Families

Any plant from the plant kingdom can be used as a source of target or template polynucleotides. Without limitation, any of the plants from the monocot class, Liliopsida or from the dicot class, Magnoliopsida are of interest. Any families from these classes that can be used in the instant invention, including without limitation:

Liliaceae, Orchidaceae, Poaceae, Iridaceae, Arecaceae, Bromeliaceae, Cyperaceae, Juncaceae, Musaceae, Ameryllidaceae, Ranunculaceae, Arecaceae; Musaceae; Brassicaceae; Rosaceae; Fabaceae; Magnoliaceae; Apiaceae; Solanaceae; Lamaiaceae; Asteraceae; Salicaceae; Cucurbitaceae; Malvaceae; and Graminaceae.

Of particular interest as plants species from the following genera, without limitation, Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

### EXAMPLES

The invention is illustrated by the following Examples. The invention is not limited by the Examples; the scope of the invention is defined only by the claims following.

### Example 1

### General Materials and Methods

Plant DNAs

Plant DNAs were isolated according to Jofuku and Goldberg (1988); “Analysis of plant gene structure”, pp. 37-66 in Plant Molecular Biology: A Practical Approach, C. H. Shaw, ed. (Oxford:IRL Press).

Oligonucleotides

Oligonucleotide primer pairs were selected from template Arabidopsis gene sequences using default parameters and the PrimerSelect 3.11 software program (Lasergene sequence analysis suite, DNASTAR, Inc., Madison, Wis.). Selected primer pairs were then used to generate PCR products utilizing genomic DNA from Brassica napus as a target plant species and polynucleotides. PCR products were either sequenced directly or cloned into E. coli using the TOPO™ TA vector cloning system according to manufacturer's guidelines (Invitrogen, Carlsbad, Calif.) . Nucleotide sequences of PCR products and/or cloned inserts were determined using an ABI PRISM@ 377 DNA Analyzer as specified by the manufacturer (PE Applied Biosystems, Foster City, Calif.) and compared to the template Arabidopsis gene sequence using default parameters and the SeqMan 3.61 software program (Lasergene sequence analysis suite, DNASTAR, Inc., Madison, Wis.). Brassica napus gene regions of greater than or equal to 17 nucleotides in length and 70% sequence identity relative to the Arabidopsis gene were selected and the nucleotide sequences translated into the corresponding amino acid sequences using standard genetic codes. Using the deduced amino acid sequences, the corresponding sequences of triplet codons of the Arabidopsis gene region, class-, family-, genera- and/or species-specific codon usage tables, oligonucleotide primer pairs were designed for use in identifying similar gene regions that would encode identical peptides in various unrelated plant genera. In all cases, the DNA sequence of a primer or its reverse complement would be identical to the sequence of triplet codons of the Arabidopsis gene sequence at nucleotide positions 1 and 2. In some cases the nucleotide at position 3 of a triplet codon would be identical to the Arabidopsis codon if that codon is preferentially used in a given plant genera and/or species as determined by published codon usage tables. In other cases, position 3 would be selected (e.g., A, G, C, T) using genera- and/or species-specific codon usage tables such that the designated nucleotide together with nucleotides in positions 1 and 2 will form a triplet codon that will encode an amino acid that is identical to that encoded by the Arabidopsis triplet codon. In some of these cases, where there is an equal probability of using one codon or another that encodes the same amino acid but differs only at position 3, then the selection of an A, G, C, or T residue will not generate a string of homopolynucleotides more than four nucleotides.

PCR

A typical PCR reaction consisted of 1-5 μg of template plant DNA, 10 pmol of each primer of a selected primer pair, and 1.25 U of Taq DNA polymerase in standard 1×PCR reaction buffer as specified by the manufacturer (Promega, Madison, Wis.). PCR reaction conditions consisted of one (1) initial cycle of denaturation at 94° C. for 7 min, thirty-five (35) cycles of denaturation at 94° C. for 1 min., primer-template annealing at 58° C. for 30 sec., synthesis at 68° C. for 4 min., and one (1) cycle of prolonged synthesis at 68° C. for 7 min.

A typical single primer PCR (SPPCR) reaction consists of 1-5 μg of template plant DNA, 10 pmol of a selected primer, and 1.25 U of Taq DNA polymerase in standard 1×PCR reaction buffer as specified by the manufacturer (Promega, Madison, Wis.). PCR reaction conditions consisted of twenty (20) cycles of denaturation at 94° C. for 30 sec., primer-template annealing at 55° C. for 30 sec., synthesis at 72° C. for 1 min., 30 sec., two cycles (2) of denaturation at 94° C. for 30 sec., primer-template annealing at 30° C. for 15 sec., 35° C. for 15 sec., 40° C. for 15 sec., 45° C. for 15 sec., 50° C. for 15 sec., 55° C. for 15 sec., 60° C. for 15 sec., 65° C. for 15 sec., and synthesis at 72° C. for 1 min., 30 sec., thirty (30) cycles of denaturation at 94° C. for 30 sec., primer-template annealing at 55° C. for 30 sec., synthesis at 72° C. for 1 min., 30 sec., followed by one (1) cycle of prolonged synthesis at 72° C. for 7 min.

Identification of Related Gene Sequences

Selected primers and/or primer pairs were used in PCR or SPPCR reactions using genomic DNAs isolated from selected plant genera to generate PCR products. Alternatively, primers and/or primer pairs could be used in RT-PCR reactions using RNA isolated from selected plant genera to generate PCR products using standard published procedures. PCR products were analyzed by agarose gel electrophoresis according to standard procedures. Specific products were extracted from agarose gels and either sequenced directly using the selected primer(s) as sequencing primers or first cloned into E. coli using the TOPO™ TA vector cloning system according to manufacturer's guidelines (Invitrogen, Carlsbad, Calif.). Cloned inserts were sequenced using an ABI PRISM™ 377 DNA Analyzer as specified by the manufacturer (PE Applied Biosystems, Foster City, Calif.). The DNA sequences obtained were then analyzed using the MapDraw 3.15 software program (Lasergene sequence analysis suite, DNASTAR, Inc., Madison, Wis.). Both nucleotide and deduced amino acid sequences were then compared to the template Arabidopsis and Brassica napus gene and amino acid sequences using default parameters and the MegAlign 3.18 software program (Lasergene sequence analysis suite, DNASTAR, Inc., Madison, Wis.) to verify gene identity.

Alternatively, selected primers and/or PCR products could be used directly as gene probes to screen plant genomic or cDNA libraries for putative related genes in various genera and/or species. Cloned inserts identified in this way would be sequenced and the nucleotide and deduced amino acid sequences analyzed as described previously.

### Example 2

### Generating Primer Sequences Using Method as Described—Computer Simulation

| | | |
|---|---|---|
| (A) | | |
| GENE: | AGAMOUS | |
| FUNCTION: | TRANSCRIPTION FACTOR | |
| DOMAIN: | MADS BOX | |
| AA SEQUENCE: | G R G K I E I K R I E | |
| Predicted NT: | GGG AGG GGC AAG AUC GAG AUC AAG CGC AUC GAG | |
| Maize | GGG AGa GGC AAG AUC GAG AUC AAG CGC AUC GAG | 32/33 |
| Rice | GGG AGG GGg AAG AUC GAG AUC AAG CGg AUC GAG | 31/33 |
| Arabidopsis | GGG AGA GGA AAG AUC GAA AUC AAA CGG AUC GAG | (M) 28/33 |
| | | (R) 29/33 |
| (B) | | |
| GENE: | APETALA1 | |
| FUNCTION: | TRANSCRIPTION FACTOR | |
| DOMAIN: | MADS BOX | |
| AA SEQUENCE: | R I E N K I N R O V T F | |
| Predicted NT: | AGG AUC GAG AAC AAG AUC AAC AAG CAG GUG ACC UUC | |
| Maize | cGG AUC GAG AAC AAG AUC AAC cGG CAG GUg ACC UUC | 33/36 |
| Rice | AGG AUC GAG AAC AAG AUC AAC cGG CAG GUG ACg UUC | 34/36 |
| Arabidopsis | AGG AUA GAG AAC AAG AUC AAA AGA CAA GUG ACA UUC | (M) 29/36 |
| | | (R) 30/36 |
| (C) | | |
| GENE: | APETALA2 | |
| FUNCTION: | TRANSCRIPTION FACTOR | |
| DOMAIN: | AP2 DOMAIN | |
| AA SEQUENCE: | G R W E S H I W D C | |
| Predicted NT: | GGC AGG UGG GAG UCC CAC AUC UGG GAC UGC | |
| Maize | GGC cGc UGG GAa UCC CAC AUC UGG GAC UGC | 27/30 |
| Arabidopsis | GGA AGA UGG GAA UCU CAU AUU UGG GAC UGU | (M) 23/30 |

### Example 3

### Specificity of Codon Adjusted Primers

The following example illustrates the specificity of codon adjusted primer pairs. Primers 1 and 2 represent primers taken directly from the sequence of the template polynucleotide. Primers 1′ and 2′ are primers wherein the sequence has been codon adjusted for monocots according to the invention. These primers were used to identify target polynucleotides from corn and rice.

| Primer 1 | | |
|---|---|---|
| AA SEQUENCE | D C G L Q V | |
| Coding Sequence: | 5′ G GAC TGT GGG AAA CAA GTT TA 3′ | |
| Primer 1 Sequence: | 5′ G GAC TGT GGG AAA CAA GTT TA 3′ | |
| Primer 1′ (Codon Adjusted Sequence): | 5′ G GAC TGC GGG AAG CAG GTG TA 3′ | 17/21 |
| % Sequence Identity to Primer 1: | 81% | |
| Primer 2 | | |
| AA SEQUENCE | K Y R G V T L | |
| Coding Sequence: | 5′ AAG TAT AGA GGT GTC ACT TTG CA 3′ | |
| Complement | 3′ TTC ATA TCT CCA CAG TGA AAC GT 5′ | |
| Primer 2 Sequence: | 5′ TG CAA AGT GAC ACC TCT ATA CTT 3′ | |
| Codon Adjusted Sequence: | 5′ AAG TAC AGG GGC GTC ACC TTG CA 3′ | |
| Complement | 3′ TTC ATG TCC CCC CAG TGG AAC GT 5′ | |
| Primer 2′ Sequence: | 5′ TG CAA GGT GAC GCC CCT GTA CTT 3′ | 19/23 |
| % Sequence Identity to Primer 2: | 83% | |

PCR was performed as described in Example 1 using genomic DNA from Arabidopsis thaliana, Oryza sativa (rice) and Zea mays (corn) as a source for the desired target polynucleotides.

Results and Conclusions

PCR-amplified products of the expected size were generated using primers 1 and 2 and Arabidopsis genomic DNA as a substrate. No products were obtained in reactions using either rice or corn genomic DNA substrate.

On the other hand, PCR-amplified products were generated using the codon adjusted primers 1′ and 2′ and corn genomic DNA as a substrate. No products were obtained in a reaction using Arabidopsis genomic DNA substrate. Together, these results demonstrate the general utility of designing codon adjusted primers for use in isolating/identifying gene orthologs from different plant families.

### Example 4

The method of the invention was used to isolate AP2-like genes from Avena sativa (oat), Oryza sativa (rice), Triticum aestivum (wheat) and Zea mays (corn). Primers 1′ and 2′ described in Example 3 were used in PCR using the conditions of Example 1 and genomic DNA from each plant as a source of target polynucleotides. The nucleotide and corresponding amino acid sequences of PCR-amplified products are shown below.

>OAT ADC GENE 489 BP
TACCTAGGTGAGCTCAAATTCCCAGCTCCAGCTCCTCCTAATTAATTTCCATCTGTTCTGTGTACTGAAGTTATTTAATTTCGTCAGGTGG
TTTCGACACCGCGCACTCGGCCGCGAGGTTATAATTAATCAAGCTTCCTAGTTTGAACTTTCAACACATACTGCTCTCTCTCGATTGGATT
GTACTAGCATCATGAACTGTACTGAAACGGGTCTTGCTCAGGGCCTACGATCGCGCGGCGATCAAGTTCCGGGGACTGGACGCCGACATCA
ACTTCAATCTGAGCGACTACGAGGAGGATCTGAAGCAGGTAACTGAATAAGATCGCTTCCTCAAATGCAGCATAGATATTATCGGTGTGTG
TGTGTCTGATGGGTGGTTGGTGGCCGGCCGGGCACTCTTGTTTTTGCCAGATGAGGAACTGGACCAAGGAGGAGTTCGTGCACATCCTCCG
CCGCCAGAGCACGGGGTTCGCGAGGGGGAGCTCA
>OAT ADC PROTEIN 65 aa
GGFDTAHSAARAYDRAAIKFRGLDADINFNLSDYEEDLKQVTNWTKEEFVHILRRQSTGFARGSS
>RICE AP2-LIKE GENE 387 BP
CCTAGGTAATTTCATCGAACACATCATCTTCCTCCTCTCAATCCAACGCGACATCGCCATGAACAATCTAACAAACACCTTCATCTTCTCC
CAAACAATCACAGGTGGATTCGACACTGCTCACGCAGCTGCAAGGTAAAGAACACATCACATCATTCATCAGAACATGAGCTCTGTGTTTG
TGAAGGAGATTGAGAGAATTGAATGATGATGGATGGATGCAGGGCGTACGACAGGGCGGCGATCAAGTTCAGGGGAGTAGAGGCTGACATC
AACTTCAACCTGAGCGACTACGAGGAGGACATGAGGCAGATGAAGAGCTTGTCCAAGGAGGAGTTCGTGCACGTTCTCCGGCGACAGAGCA
CCGGCTTCTCCCGCGGCAGCTCA
>RICE ADC PROTEIN 65 aa
GGFDTAHAAARAYDRAAIKF RGVEADINFNLSDYEEDMRQMKSLSKEEFVHVLRRQSTGFSRGSS
>WHEAT ADC GENE 477 BP
CTTGGGTGGGTTTGACACTGCACATGCTGCTGCAAGGTACGTACAAATTTAATTAAGCACGTACGCAGTACATAATTGTGATGTGATCATC
ACCTGAACCACCTGTACTGCAACTCTGAAGTTATGTCTCCACTCTGTTCATTTCACCGTGCCAAATTGACCTTGGGATGTTCCGCAGGGCG
TACGATCGAGCGGCGATCAAGTTCCGCGGCGTCGACGCCGACATAAACTTCAACCTCAGCGACTACGAGGACGACATGAAGCAGGTGATCA
GCAAAGCCACCAACCAGTGTTCCTCATCCAACCAAATTATTCAGATGCAGAGTGCATTAGTACTGTTGTTGAAACTGATGAACTGAAGAAA
TTCTGACTGTGTGTTGKTTGGTGGATGATCTGGATCAGATGAAGGGCCTGTCCAAGGAGGAGTTCGTGCACGTGCTGCGGCGGCAGAGCGC
CGGCTTCTCGCGGGGCAGCTCC
>WHEAT ADC PROTEIN 65 aa
GGFDTAHAAARAYDRAAIKFRGVDADINFNLSDYEDDMKQVKGLSKEEFVHVLRRQSAGFSRGSS
>MAIZE ADC GENE 489 BP
CTTAGGTGAGCAGCAATAAGCAGATCGATCTGCAGCATAAATTTCCCGTTATTAACTAGTTCGTGATCTCGATCGAATGGCCTAATTAACC
GATTCGGTGAT CTGGCCGATGGCCAATCTACGCAGGTGGATTCGACACTGCTCATGCCGCTGCAAGGTAACGATCAATCCATCCATCCACC
CTTGTCTAGCTACCCCACCGACCGGCCGGATTAATGGACCGCTAGTTCTCGGGACGGGCTTGCTGCAGGGCGTACGACCGAGCGGCGATCA
AGTTCCGCGGCGTCGACGCCGACATAAACTTCAACCTCAGCGACTACGACGACGATATGAAGCAGGTACATACACGAGTGTTGTT GCAGCT
AGCACCGACTGAAACATCTGCTGAACGTACACTCATGGCCTGTGCACCAGATGAAGAGCCTGTCCAAGGAGGAGTTCGTGCACGCCCTGCG
GCGGCAGAGCACCGGCTTCTCCCGTGGCAGCTCC
>MAIZE ADC PROTEIN 65 aa
GGFDTAHAAARAYDRAAIKFRGVDADINFNLSDYDDDMKQVKSLSKEEFVHALRRQSTGFSRGSS

### Example 5

### Use of Short Codon Adjusted Primers

Oligonucleotides

Codon adjusted oligonucleotides were designed as described previously. Derivatives of oligonucleotide 2′ were generated as shown above and used as primers in combination with oligonucleotide 1′ in PCR reactions using plant genomic DNA from Zea mays (corn), Avena sativa (oat), and Triticum aestivum (wheat) as a source of target polynucleotides.

PCR

A typical PCR reaction consisted of 1-5 μg of target plant DNA, 10 pmol of primer 1′ and 10 pmol of a derivitive of primer 2′, and 1.25 U of Taq DNA polymerase in standard 1×PCR reaction buffer as specified by the manufacturer (Promega, Madison, Wis.). PCR reaction conditions consisted of five cycles (5) of denaturation at 94oC for 2 minutes, 94oC for 30 sec., primer-template annealing at 65oC for 15 sec., 60oC for 15 sec., 55oC for 15 sec., 50oC for 15 sec., 45oC for 15 sec., 40oC for 15 sec., and synthesis at 68oC for 1 min., 30 sec., and twenty (20) cycles of denaturation at 94oC for 30 sec., primer-template annealing at 55oC for 30 sec., synthesis at 72oC for 1 min., 30 sec., thirty (30) cycles of denaturation at 94oC for 30 sec., primer-template annealing at 50oC for 30 sec., synthesis at 68oC for 1 min., followed by one (1) cycle of prolonged synthesis at 68oC for 7 min.

| | |
|---|---|
| Primer 1 | |
| AA SEQUENCE | D C G L Q V |
| Coding Sequence: | 5′ C GAC TGT GGG AAA CAA GTT TA 3′ |
| Primer Sequence: | 5′ G GAC TGT GGG AAA CAA GTT TA 3′ |
| Primer 1′ (Codon Adjusted Sequence): | 5′ G GAC TGC GGG AAG CAG GTG TA 3′ |
| Primer 2 | |
| AA SEQUENCE | K Y R G V T L |
| Coding Sequence: | 5′ AAG TAT AGA GGT GTC ACT TTG CA 3′ |
| Complement | 3′ TTC ATA TCT CCA CAG TCA AAC GT 5′ |
| Primer 2 Sequence: | 5′ TG CAA AGT GAC ACC TCT ATA CTT 3′ |
| Codon Adjusted Sequence: | 5′ AAG TAC AGG GGC GTC ACC TTG CA 3′ |
| Complement | 3′ TTC ATG TCC CCG CAG TGG AAC GT 5′ |
| Primer 2′ Sequence: | 5′ TG CAA GGT GAC GCC CCT GTA CTT 3′ |
| RISZU2′-1 (5 CODONS) | 5′ G CAA GGT GAC GCC CCT GT 3′ |
| RISZU2′-2 (5 CODONS) | 5′ GGT GAC GCC CCT GTA CT 3′ |
| RISZU2′-3 (4 CODONS) | 5′ GT GAC GCC CCT GTA CT 3′ |
| RISZU2′-4 (3 CODONS) | 5′ GT GAC GCC CCT GT 3′ |

Results and Conclusions

As described in Methods, primer 2′ derivitives vary in length from 15-18 bp that could encode a peptide of 4-5 amino acids in length. FIG. 3 shows that PCR-amplified products were generated using primer 1′ and primer 2′ derivitives 1, 2, and 3 and all three genomic DNAs as a source of target polynucleotides.

These results demonstrate that the method as described can utilize conserved regions of greater than or equal to 4 amino acids in length for use in isolating/identifying gene orthologs from different plant families.

What is claimed:

1. A method for isolating a from a target plant species a target polynucleotide encoding a target polypeptide comprising a conserved region exhibiting at least 70% sequence identity to a conserved region of template polypeptide that is encoded by a template polynucleotide from a template plant species, comprising:
(a) identifying an amino acid sequence of a conserved region in the template polypeptide;
(b) generating an oligonucleotide comprising a sequence wherein the sequence or its reverse complement comprises at least four codons that encode a portion of the amino acid sequence of (a), wherein
(i) the sequence of the first and second positions of at least three of the codons is the same as the corresponding nucleotides in nucleotides in the template polynucleotide;
(ii) the nucleotide at the third position of the codons of (i) is the nucleotide of the third position of the most preferred codon of the target plant class for the desired amino acid;
(c) contacting the oligonucleotide with a composition comprising the target polynucleotide under conditions that permit hybridization of the oligonucleotide to the target polynucleotide to form a duplex; and
(d) isolating the duplex.

2. The method of claim 1, wherein the oligonucleotide does not contain a homopolymer of more than four guanine or cytosine residues.

3. The method of claim 1, wherein the oligonucleotide does not contain a homopolymer of more than four residues.

4. The method of claim 1, wherein the oligonucleotide of step (b) wherein the sequence or its reverse complement further comprises at least one codon wherein
(i) the sequence of the first and second position of the codon is the same as the corresponding nucleotides in the template polynucleotide;
(ii) the sequence of the third position of the codon of step (I) is the same as the nucleotide of the third position of the second most preferred codon of the target plant species for the desired amino acid; and
(iii) the oligonucleotide is not degenerate.

5. The method of claim 1, wherein the target polynucleotide is from a monocot plant and the template polynucleotide is from a dicot plant.

6. The method of claim 4, wherein the template polynucleotide is from Arabidopsis.

7. The method of claim 5, wherein the third position of each codon is either a guanosine or cytosine.

8. The method of claim 2, wherein both the template and target polynucleotides are from dicot plants.

9. The method of claim 8, wherein the template polynucleotide is from Arabidopsis.

10. The method of claim 9, wherein the third position of each codon is either an adenosine or thymidine.

11. The method of claim 1, wherein the template polynucleotide is from a monocot plant and the target polynucleotide is from a dicot plant.

12. The method of claim 1, wherein both the template and target polynucleotides are from monocot plants.

13. The method of claim 11 or 12, wherein the template polynucleotide is from corn.

14. The method of claim 12, wherein the target polynucleotide is from corn.

15. The method of claim 1, wherein step (a) comprises aligning polynucleotides of plants within a family and identifying a portion of the template polynucleotide that exhibits at least 70% sequence identity to a portion of a polynucleotide from a plant of a genus closely related to the plant from which the template polynucleotide originates.

16. The method of claim 1, wherein step (a) comprises identifying the primary sequence in a region of the template polypeptide sequence that forms a secondary structure.

17. The method of claim 16, wherein the secondary structure is a helix or a beta sheet.

18. The method of claim 1, wherein the conserved region is a motif or functional domain.

19. The method of claim 1, wherein step (a) comprises identifying the primary sequence in a region of the template polypeptide that is repeated.

20. The method of claim 1, wherein the oligonucleotide comprises from 6 to 11 codons.

21. The method of claim 1, wherein step (c) further includes contacting the composition comprising the target polynucleotide with a second oligonucleotide, wherein the second oligonucleotide is a degenerate oligonucleotide encoding a second portion of the conserved region.

22. A method of isolating a target polynucleotide encoding a conserved region in a template polypeptide encoded by a template polynucleotide comprising:
(a) identifying the amino acid sequence of the conserved region in the template polypeptide;
(b) generating an oligonucleotide comprising a sequence wherein the sequence or its reverse complement comprises at least four codons that encode a portion of the conserved region of step (a), wherein
(i) the sequence of the first and second positions of at least three codons is the same as the corresponding nucleotides in the template polynucleotide;
(ii) the nucleotide of the third position of those six codons is the same nucleotide in the third position of the most preferred codon of the target plant species for the desired amino acid;
(iii) the oligonucleotide does not comprise homopolymers of more than four nucleotides; and
(iv) the oligonucleotide is not degenerate;
(c) contacting the oligonucleotide with a composition comprising the target polynucleotide under conditions that permit hybridization of the oligonucleotide to the target polynucleotide to form a duplex;
(d) contacting the duplex of step (c) with a thermostable polymerase under conditions to elongate the oligonucleotide of step (b); and
(e) isolating the elongation product of step (d).

23. A method for identifying a target polynucleotide encoding a conserved region in a template polypeptide encoded by a template polynucleotide comprising:
(a) identifying the amino acid sequence of the conserved region in the template polypeptide;
(b) generating an oligonucleotide comprising a sequence wherein the sequence or its reverse complement comprises four codons that encode a portion of the conserved region of step (a), wherein
(i) the sequence of the first and second positions of at least three codons is the same as the corresponding nucleotides in the template polynucleotide;
(ii) the nucleotide of the third position of those six codons is the same nucleotide in the third position of the most preferred codon of the target plant species for the desired amino acid;
(iii) the oligonucleotide does not comprise homopolymers of more than four nucleotides; and
(iv) the oligonucleotide is not degenerate;
(c) contacting the oligonucleotide with a composition comprising the target polynucleotide under conditions that permit hybridization of the oligonucleotide to the target polynucleotide to form a duplex;
(d) contacting the duplex of step (c) with a thermostable polymerase under conditions to elongate the oligonucleotide of step (b); and
(e) determining the nucleotide sequence of the elongation product of step (d).

24. A method of isolating a target polynucleotide encoding a polypeptide of a conserved region in a template polypeptide encoded by a template polynucleotide, comprising:
(a) identifying the amino acid sequence of the conserved region in the template polypeptide;
(b) generating a first oligonucleotide comprising a sequence wherein the sequence or its reverse complement comprises four codons that encode a first portion of the conserved region of step (a), wherein
(i) the sequence of the first and second position of at least three codons is the same as the corresponding nucleotides in the template polynucleotide;
(ii) the nucleotide of the third position of the codons of step (i) is the same as the nucleotide in the third position of the most preferred codon of the target plant species for the desired amino acid;
(iii) the oligonucleotide does not comprise homopolymers of more than four nucleotides; and
(iv) the oligonucleotide is not degenerate;
(c) generating a second oligonucleotide wherein its sequence or its reverse complement comprises four codons that encode a second portion of the conserved region of step (a), wherein
(i) the sequence of the first and second position of at least three codons is the same as the corresponding position in the template polynucleotide;
(ii) the nucleotide of the third position of those codons is the same as the nucleotide of the third position of the most preferred codon of the target plant species for the desired amino acid;
(iii) the oligonucleotide does not comprise homopolymers of more than four nucleotides; and
(iv) the oligonucleotide is not degenerate;
(d) contacting the first and second oligonucleotides with a composition comprising the target polynucleotide under conditions that permit hybridization of at least one of the oligonucleotides and the target polynucleotide to form a duplex;
(e) contacting the duplex of step (d) with a thermostable polymerase under conditions to elongate the at least one hybridized oligonucleotide;
(f) generating a strand complementary to the elongation product of step (e); and
(g) isolating the product of step (d).

25. The method of claim 24, wherein the two oligonucleotide sequences or their reverse complements encode portions of the conserved region of step (a) that are separated by at least 30 amino acids.

26. The method of claim 24, wherein the two oligonucleotide sequences or their reverse complement encode between 6 to 11 amino acids of the conserved region of step (a).

27. The method of claim 24, wherein the product of step (f) is inserted into a vector.

28. A method for identifying a target polynucleotide encoding a polypeptide of a conserved region in a template polypeptide encoded by a template polynucleotide, comprising:
(a) identifying the amino acid sequence of the conserved region in the template polypeptide;
(b) generating a first oligonucleotide comprising a sequence wherein the sequence or its reverse complement comprises four codons that encode a first portion of the conserved region of step (a), wherein
(i) the sequence of the first and second position of at least three codons is the same as the corresponding nucleotides in the template polynucleotide;
(ii) the nucleotide of the third position of the codons of step (i) is the same as the nucleotide in the third position of the most preferred codon of the target plant species for the desired amino acid;
(iii) the oligonucleotide does not comprise homopolymers of more than four nucleotides; and
(iv) the oligonucleotide is not degenerate;
(c) generating a second oligonucleotide wherein its sequence or its reverse complement comprises four codons that encode a second portion of the conserved region of step (a), wherein
(i) the sequence of the first and second position of at least three codons is the same as the corresponding position in the template polynucleotide;
(ii) the nucleotide of the third position of those codons is the same as the nucleotide of the third position of the most preferred codon of the target plant species for the desired amino acid;
(iii) the oligonucleotide does not comprise homopolymers of more than four nucleotides; and
(iv) the oligonucleotide is not degenerate;
(d) contacting the first and second oligonucleotides with a composition comprising the target polynucleotide under conditions that permit hybridization of at least one of the oligonucleotides and the target polynucleotide to form a duplex;
(e) contacting the duplex of step (d) with a thermostable polymerase under conditions to elongate the at least one hybridized oligonucleotide;
(f) generating a strand complementary to the elongation product of step (e); and
(g) determining the nucleotide sequence of the product of step (f).

29. A method for selecting a nucleotide sequence of an oligonucleotide primer for a polymerase chain reaction comprising:
(a) selecting a nucleotide sequence encoding a desired amino acid sequence from a template organism, or the complement thereof;
(b) selecting for the nucleotide of the third position of each codon the preferred codon for a target organism, provided said nucleotide is guanine or cytosine;
(c) if the nucleotide of the third position of the preferred codon is adenine or thymine, then substituting either a guanine or cytosine, selecting guanine or cytosine to avoid introducing a poly-guanylate or polycytidylate sequence of more than four residues;
wherein said desired amino acid sequence is encoded by one reading frame, or a portion thereof, of the nucleotide sequence of said primer or the complement thereof.

30. A method for preparing an oligonucleotide primer for a polymerase chain reaction comprising:
(a) selecting a nucleotide sequence encoding a desired amino acid sequence from a template organism, or the complement thereof;
(b) selecting for the nucleotide of the third position of each codon the preferred codon for a target organism, provided said nucleotide is guanine or cytosine;
(c) if the nucleotide of the third position of the preferred codon is adenine or thymine, then substituting either a guanine or cytosine, selecting guanine or cytosine to avoid introducing a poly-guanylate or polycytidylate sequence of more than four residues; and
(d) synthesizing said oligonucleotide primer, wherein said desired amino acid sequence is encoded by one reading frame, or a portion thereof, of the nucleotide sequence of said primer or the complement thereof.

31. A method for cloning a nucleic acid comprising:
(a) selecting an upstream nucleotide sequence encoding a first desired amino acid sequence from a template organism and a downstream nucleotide sequence encoding a second desired amino acid sequence;
(b) for each of said upstream and downstream nucleotide sequences, selecting for the nucleotide of the third position of each codon the preferred codon for a target organism, provided said nucleotide is guanine or cytosine;
(c) if the nucleotide of the third position of the preferred codon is adenine or thymine, then substituting either a guanine or cytosine, selecting guanine or cytosine to avoid introducing a poly-guanylate or polycytidylate sequence of more than four residues.

32. The method of claim 31, further comprising:
(d) synthesizing an upstream oligonucleotide primer, or a portion thereof according to steps (b) and (c).

33. The method of claim 32, further comprising:
(e) performing a polymerase chain reaction using said upstream and downstream primers and a template comprising a nucleic acid sample obtained from said target organism.

34. The method of claim 33, further comprising:
(f) using the product of said polymerase chain reaction of step (e) as a probe to screen a library prepared from nucleic acids obtained from said target organism.

35. The method of claim 33, further comprising:
(f′) inserting the product of the polymerase chain reaction of step (e) into a vector.

36. The method of any one of claims 30-35, wherein said template organism is a dicot and said target organism is a monocot or wherein said template organism is a monocot and said target organism is a dicot.

37. A method for isolating a target polynucleotide encoding a target polypeptide comprising a conserved region of a template polypeptide that is encoded by a template polynucleotide, comprising:
(a) identifying an amino acid sequence of a conserved region in the template polypeptide;
(b) generating an oligonucleotide comprising a sequence wherein the sequence or its reverse complement comprises at least four codons that encode a portion of the amino acid sequence of (a), wherein
(i) the sequence of the first and second positions of at least three of the codons is the same as the corresponding nucleotides in nucleotides in the template polynucleotide;
(ii) the nucleotide at the third position of the codons of (i) is the nucleotide of the third position of the most preferred codon of the target plant species for the desired amino acid;
(c) contacting the oligonucleotide with a composition comprising the target polynucleotide under conditions that permit hybridization of the oligonucleotide to the target polynucleotide to form a duplex; and
(d) generating a single strand polynucleotide.

38. A method for isolating a from a target plant species a target polynucleotide encoding a target polypeptide comprising a conserved region exhibiting at least 70% sequence identity to a conserved region of template polypeptide that is encoded by a template polynucleotide from a template plant species, comprising:
(a) identifying an amino acid sequence of a conserved region in the template polypeptide;
(b) generating an oligonucleotide comprising a sequence wherein the sequence or its reverse complement comprises at least four codons that encode a portion of the amino acid sequence of (a), wherein
(i) the sequence of the first and second positions of at least three of the codons is the same as the corresponding nucleotides in nucleotides in the template polynucleotide;
(ii) the nucleotide at the third position of the codons of (i) is the nucleotide of the third position of the most preferred codon of the plant family of target plant species for the desired amino acid;
(c) contacting the oligonucleotide with a composition comprising the target polynucleotide under conditions that permit hybridization of the oligonucleotide to the target polynucleotide to form a duplex; and
(d) isolating the duplex.

39. A method for isolating a from a target plant species a target polynucleotide encoding a target polypeptide comprising a conserved region exhibiting at least 70% sequence identity to a conserved region of template polypeptide that is encoded by a template polynucleotide from a template plant species, comprising:
(a) identifying an amino acid sequence of a conserved region in the template polypeptide;
(b) generating an oligonucleotide comprising a sequence wherein the sequence or its reverse complement comprises at least four codons that encode a portion of the amino acid sequence of (a), wherein
(i) the sequence of the first and second positions of at least three of the codons is the same as the corresponding nucleotides in nucleotides in the template polynucleotide;
(ii) the nucleotide at the third position of the codons of (i) is the nucleotide of the third position of the most preferred codon of the plant genera for the target plant species for the desired amino acid;
(c) contacting the oligonucleotide with a composition comprising the target polynucleotide under conditions that permit hybridization of the oligonucleotide to the target polynucleotide to form a duplex; and
(d) isolating the duplex.

40. A method for isolating a from a target plant species a target polynucleotide encoding a target polypeptide comprising a conserved region exhibiting at least 70% sequence identity to a conserved region of template polypeptide that is encoded by a template polynucleotide from a template plant species, comprising:
(a) identifying an amino acid sequence of a conserved region in the template polypeptide;
(b) generating an oligonucleotide comprising a sequence wherein the sequence or its reverse complement comprises at least four codons that encode a portion of the amino acid sequence of (a), wherein
(i) the sequence of the first and second positions of at least three of the codons is the same as the corresponding nucleotides in nucleotides in the template polynucleotide;
(ii) the nucleotide at the third position of the codons of (i) is the nucleotide of the third position of the most preferred codon of the plant species of the target plant species for the desired amino acid;
(c) contacting the oligonucleotide with a composition comprising the target polynucleotide under conditions that permit hybridization of the oligonucleotide to the target polynucleotide to form a duplex; and
(d) isolating the duplex.


SEQUENCE LISTING (48 sequences)

SEQ ID NO: 1 - protein, 11 aa, Arabidopsis and Brassica napus
GRGKIEIKRIE

SEQ ID NO: 2 - DNA, 33 bp, Arabidopsis and Brassica napus
gggaggggcaagaucgagaucaagcgcaucgag

SEQ ID NO: 3 - DNA, 33 bp, Maize
gggagaggcaagaucgagaucaagcgcaucgag

SEQ ID NO: 4 - DNA, 33 bp, Oryza sativa
gggagggggaagaucgagaucaagcggaucgag

SEQ ID NO: 5 - DNA, 33 bp, Arabidopsis
gggagaggaaagaucgaaaucaaacggaucgag

SEQ ID NO: 6 - protein, 12 aa, Arabidopsis and Brassica napus
RIENKINRQVTF

SEQ ID NO: 7 - DNA, 36 bp, Arabidopsis and Brassica napus
aggaucgagaacaagaucaacaagcaggugaccuuc

SEQ ID NO: 8 - DNA, 36 bp, Maize
cggaucgagaacaagaucaaccggcaggugaccuuc

SEQ ID NO: 9 - DNA, 36 bp, Oryza sativa
aggaucgagaacaagaucaaccggcaggugacguuc

SEQ ID NO: 10 - DNA, 36 bp, Arabidopsis
aggauagagaacaagaucaaaagacaagugacauuc

SEQ ID NO: 11 - protein, 10 aa, Arabidopsis and Brassica napus
GRWESHIWDC

SEQ ID NO: 12 - DNA, 30 bp, Arabidopsis and Brassica napus
ggcaggugggagucccacaucugggacugc

SEQ ID NO: 13 - DNA, 30 bp, Maize
ggccgcugggaaucccacaucugggacugc

SEQ ID NO: 14 - DNA, 30 bp, Arabidopsis
ggaagaugggaaucucauauuugggacugu

SEQ ID NO: 15 - protein, 6 aa, Zea mays and Oryza sativa
DCGLQV

SEQ ID NO: 16 - DNA, 21 bp, Zea mays and Oryza sativa
ggactgtgggaaacaagttta

SEQ ID NO: 17 - DNA, 21 bp, Zea mays and Oryza sativa
ggactgtgggaaacaagttta

SEQ ID NO: 18 - DNA, 21 bp, Zea mays and Oryza sativa
ggactgcgggaagcaggtgta

SEQ ID NO: 19 - protein, 7 aa, Zea mays and Oryza sativa
KYRGVTL

SEQ ID NO: 20 - DNA, 23 bp, Zea mays and Oryza sativa
aagtatagaggtgtcactttgca

SEQ ID NO: 21 - DNA, 23 bp, Zea mays and Oryza sativa
tgcaaagtgacacctctatactt

SEQ ID NO: 22 - DNA, 23 bp, Zea mays and Oryza sativa
tgcaaagtgacacctctatactt

SEQ ID NO: 23 - DNA, 23 bp, Zea mays and Oryza sativa
aagtacaggggcgtcaccttgca

SEQ ID NO: 24 - DNA, 23 bp, Zea mays and Oryza sativa
tgcaaggtgacgcccctgtactt

SEQ ID NO: 25 - DNA, 23 bp, Zea mays and Oryza sativa
tgcaaggtgacgcccctgtactt

SEQ ID NO: 26 - DNA, 489 bp, Avena sativa ADC Gene
tacctaggtgagctcaaattcccagctccagctcctcctaattaatttccatctgttctgtgtactgaagttatttaatttcgtcaggtggtttcgacaccgcgcactcggccgcgaggttataattaatcaagcttcctagtttgaactttcaacacatactgctctctctcgattggattgtactagcatcatgaactgtactgaaacgggtcttgctcagggcctacgatcgcgcggcgatcaagttccggggactggacgccgacatcaacttcaatctgagcgactacgaggaggatctgaagcaggtaactgaataagatcgcttcctcaaatgcagcatagatattatcggtgtgtgtgtgtctgatgggtggttggtggccggccgggcactcttgtttttgccagatgaggaactggaccaaggaggagttcgtgcacatcctccgccgccagagcacggggttcgcgagggggagctca

SEQ ID NO: 27 - protein, 65 aa, Avena sativa ADC PROTEIN
GGFDTAHSAARAYDRAAIKFRGLDADINFNLSDYEEDLKQVTNWTKEEFVHILRRQSTGFARGSS

SEQ ID NO: 28 - DNA, 387 bp, Oryza sativa AP2-LIKE GENE
cctaggtaatttcatcgaacacatcatcttcctcctctcaatccaacgcgacatcgccatgaacaatctaacaaacaccttcatcttctcccaaacaatcacaggtggattcgacactgctcacgcagctgcaaggtaaagaacacatcacatcattcatcagaacatgagctctgtgtttgtgaaggagattgagagaattgaatgatgatggatggatgcagggcgtacgacagggcggcgatcaagttcaggggagtagaggctgacatcaacttcaacctgagcgactacgaggaggacatgaggcagatgaagagcttgtccaaggaggagttcgtgcacgttctccggcgacagagcaccggcttctcccgcggcagctca

SEQ ID NO: 29 - protein, 65 aa, Oryza sativa ADC PROTEIN
GGFDTAHAAARAYDRAAIKFRGVEADINFNLSDYEEDMRQMKSLSKEEFVHVLRRQSTGFSRGSS

SEQ ID NO: 30 - DNA, 477 bp, Triticum aestivum ADC GENE
cttgggtgggtttgacactgcacatgctgctgcaaggtacgtacaaatttaattaagcacgtacgcagtacataattgtgatgtgatcatcacctgaaccacctgtactgcaactctgaagttatgtctccactctgttcatttcaccgtgccaaattgaccttgggatgttccgcagggcgtacgatcgagcggcgatcaagttccgcggcgtcgacgccgacataaacttcaacctcagcgactacgaggacgacatgaagcaggtgatcagcaaagccaccaaccagtgttcctcatccaaccaaattattcagatgcagagtgcattagtactgttgttgaaactgatgaactgaagaaattctgactgtgtgttgkttggtggatgatctggatcagatgaagggcctgtccaaggaggagttcgtgcacgtgctgcggcggcagagcgccggcttctcgcggggcagctcc

SEQ ID NO: 31 - protein, 65 aa, Triticum aestivum ADC PROTEIN
GGFDTAHAAARAYDRAAIKFRGVDADINFNLSDYEDDMKQVKGLSKEEFVHVLRRQSAGFSRGSS

SEQ ID NO: 32 - DNA, 489 bp, Zea mays ADC GENE
cttaggtgagcagcaataagcagatcgatctgcagcataaatttcccgttattaactagttcgtgatctcgatcgaatggcctaattaaccgattcggtgatctggccgatggccaatctacgcaggtggattcgacactgctcatgccgctgcaaggtaacgatcaatccatccatccacccttgtctagctaccccaccgaccggccggattaatggaccgctagttctcgggacgggcttgctgcagggcgtacgaccgagcggcgatcaagttccgcggcgtcgacgccgacataaacttcaacctcagcgactacgacgacgatatgaagcaggtacatacacgagtgttgttgcagctagcaccgactgaaacatctgctgaacgtacactcatggcctgtgcaccagatgaagagcctgtccaaggaggagttcgtgcacgccctgcggcggcagagcaccggcttctcccgtggcagctcc

SEQ ID NO: 33 - protein, 65 aa, Zea mays ADC PROTEIN
GGFDTAHAAARAYDRAAIKFRGVDADINFNLSDYDDDMKQVKSLSKEEFVHALRRQSTGFSRGSS

SEQ ID NO: 34 - protein, 6 aa, Zea mays, Avena sativa and Triticum aestivum
DCGLQV

SEQ ID NO: 35 - DNA, 21 bp, Zea mays, Avena sativa and Triticum aestivum
ggactgtgggaaacaagttta

SEQ ID NO: 36 - DNA, 21 bp, Zea mays, Avena sativa and Triticum aestivum
ggactgtgggaaacaagttta

SEQ ID NO: 37 - DNA, 21 bp, Zea mays, Avena sativa and Triticum aestivum
ggactgcgggaagcaggtgta

SEQ ID NO: 38 - protein, 7 aa, Zea mays, Avena sativa and Triticum aestivum
KYRGVTL

SEQ ID NO: 39 - DNA, 23 bp, Zea mays, Avena sativa and Triticum aestivum
aagtatagaggtgtcactttgca

SEQ ID NO: 40 - DNA, 23 bp, Zea mays, Avena sativa and Triticum aestivum
tgcaaagtgacacctctatactt

SEQ ID NO: 41 - DNA, 23 bp, Zea mays, Avena sativa and Triticum aestivum
tgcaaagtgacacctctatactt

SEQ ID NO: 42 - DNA, 23 bp, Zea mays, Avena sativa and Triticum aestivum
aagtacaggggcgtcaccttgca

SEQ ID NO: 43 - DNA, 23 bp, Zea mays, Avena sativa and Triticum aestivum
tgcaaggtgacgcccctgtactt

SEQ ID NO: 44 - DNA, 23 bp, Zea mays, Avena sativa and Triticum aestivum
tgcaaggtgacgcccctgtactt

SEQ ID NO: 45 - DNA, 18 bp, Zea mays, Avena sativa and Triticum aestivum
gcaaggtgacgcccctgt

SEQ ID NO: 46 - DNA, 17 bp, Zea mays, Avena sativa and Triticum aestivum
ggtgacgcccctgtact

SEQ ID NO: 47 - DNA, 16 bp, Zea mays, Avena sativa and Triticum aestivum
gtgacgcccctgtact

SEQ ID NO: 48 - DNA, 13 bp, Zea mays, Avena sativa and Triticum aestivum
gtgacgcccctgt